(19) United States
(12) Patent Application Publication
(10) Pub. No.: US 2022/0395197 A1
(43) Pub. Date: Dec. 15, 2022

(54) WALKING STATE MEASUREMENT SYSTEM, WALKING STATE MEASUREMENT METHOD AND NON-TRANSITORY COMPUTER READABLE MEDIUM

(71) Applicant: TOYOTA JIDOSHA KABUSHIKI KAISHA, Toyota-shi (JP)

(72) Inventors: Yoshihiro TAKIGUCHI, Toyota-shi (JP); Yoshiaki KATO, Miyoshi-shi (JP)

(73) Assignee: TOYOTA JIDOSHA KABUSHIKI KAISHA, Toyota-shi (JP)

(21) Appl. No.: 17/700,581

(22) Filed: Mar. 22, 2022

(30) Foreign Application Priority Data
Jun. 15, 2021 (JP) 2021-099240

(51) Int. Cl. A61B 5/11; A61H 1/02; A61B 5/00

(52) U.S. Cl. CPC A61B 5/1118; A61B 5/112; A61H 1/0262; A61B 5/0077

(57) ABSTRACT

A walking state measurement system includes: an endless belt that defines a walking surface on which a subject walks and that travels along a walking front-rear direction; a marker provided on at least the walking surface along a circumferential direction of the belt; a first camera for taking an image of the subject from front, the first camera taking an image of the marker on the walking surface from above to generate a first image; a second camera for taking an image of the subject from front, the second camera taking an image of the marker on the walking surface from above to generate a second image; and a correction processing unit that corrects an image generated by at least either the first camera or the second camera based on a position of an image area of the marker included in each of the first image and the second image.

### CROSS-REFERENCE TO RELATED APPLICATION

This application claims priority to Japanese Patent Application No. 2021-099240 filed on Jun. 15, 2021, incorporated herein by reference in its entirety.

### BACKGROUND

### 1. Technical Field

The present disclosure relates to a walking state measurement system.

### 2. Description of Related Art

A rehabilitation (rehab) training system has been developed for rehab patients to train their walking motion. For example, Japanese Unexamined Patent Application Publication No. 2017-035220 (JP 2017-035220 A) discloses a walking training device that assists the motion of joints of a patient via a walking assist device in accordance with the walking state of a user who is wearing the walking assist device on his/her leg. In recent years, a technique for a rehab training system has been developed in which a three-dimensional position of a trainee's leg is measured using a red-green-blue (RGB) camera and a depth camera to determine the walking state of the patient walking on a treadmill.

Here, when angle deviation or position deviation of the camera itself occurs in at least one of the RGB camera and the depth camera, the measurement accuracy of the three-dimensional position is lowered, the walking state of the patient cannot be accurately determined, and eventually the motion of the joint cannot be properly assisted. In order to suppress this, a marker is provided in the booth frame of the rehab training system and the positions of the marker in respective images of the RGB camera and the depth camera are collated to correct the angle deviation and the position deviation.

### SUMMARY

However, when the marker is located at an end of the field of view of the camera, it is easily affected by distortion of the peripheral portion of the lens, and the marker to be collated is easily erroneously recognized. The present disclosure has been made to solve such an issue, and provides a walking state measurement system that suppresses erroneous recognition of a marker in calibration between a plurality of cameras.

A walking state measurement system according to an aspect of the present disclosure includes: an endless belt that defines a walking surface on which a subject walks and that travels along a walking front-rear direction; a marker provided on at least the walking surface of the belt along a circumferential direction of the belt; a first camera for taking an image of the subject from front, the first camera taking an image of the marker on the walking surface from above to generate a first image; a second camera for taking an image of the subject from front, the second camera taking an image of the marker on the walking surface from above to generate a second image; and a correction processing unit that corrects an image generated by at least one of the first camera and the second camera based on a position of an image area of the marker included in each of the first image and the second image. The marker provided along the circumferential direction of the belt allows the camera to recognize the marker even when the belt rotates. Further, the marker is provided on the walking surface, that is, toward the center of the field of view of the camera. Therefore, it is possible to reduce the influence of distortion in the peripheral portion of the lens.

In the first image, pixel counts from both ends of the first image to both ends of the marker in a walking right-left direction may be equal to or more than a first threshold value. In the second image, pixel counts from both ends of the second image to both ends of the marker in the walking right-left direction may be equal to or more than a second threshold value. With this, the marker is located toward the center of the field of view of the camera.

In the first image, pixel counts from both ends of the first image to both ends of at least one marker in the walking front-rear direction may be equal to or more than a third threshold value. In the second image, pixel counts from both ends of the second image to both ends of at least one marker in the walking front-rear direction may be equal to or more than a fourth threshold value. With this, the marker is located toward the center of the field of view of the camera.

Further, markers may be arranged at an interval of a predetermined distance or more over an entire circumference of the belt. With this, it is possible to suppress erroneous recognition of the shape between a plurality of cameras. In addition, since feature points of the marker are positioned inward of the most end portions of the belt, it is possible to reduce the influence of distortion in the peripheral portion of the lens. Even when any of the cameras cannot sufficiently capture the most end portions of the belt, the feature points of the marker can be detected.

The markers on the walking surface may have different shapes or colors from each other. With this, it is possible to suppress the problem that calibration between the cameras is not performed correctly due to erroneous recognition of correspondence of the feature points of the markers between the plurality of cameras. The markers may constitute a pattern with at least a half circumference of the belt being one cycle.

The marker may be provided endlessly over an entire circumference of the belt. This makes it easier for the camera to capture the marker even when the belt rotates.

According to the present disclosure, it is possible to provide a walking state measurement system that suppresses erroneous recognition of markers in calibration between a plurality of cameras.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a schematic perspective view of a walking training system according to a first embodiment;

FIG. 2 is a schematic perspective view showing a configuration example of a walking assist device;

FIG. 3 is a diagram illustrating an operation of a system control unit according to the first embodiment;

FIG. 4A is a perspective view showing a belt and a field of view of a camera according to the first embodiment;

FIG. 4B is a perspective view showing the belt and the field of view of the camera according to the first embodiment;

FIG. 5A is a diagram showing an example of a first image according to the first embodiment;

FIG. 5B is a diagram showing an example of a second image according to the first embodiment;

FIG. 6 is a block diagram showing a functional configuration of an information processing device according to the first embodiment;

FIG. 7 is a flowchart showing an information processing procedure of the information processing device according to the first embodiment;

FIG. 8 is a diagram illustrating a first example of a marker according to a second embodiment;

FIG. 9 is a diagram illustrating a second example of the marker according to a second embodiment;

FIG. 10 is a diagram illustrating a third example of the marker according to the second embodiment;

FIG. 11 is a diagram illustrating a fourth example of the marker according to the second embodiment;

FIG. 12 is a diagram illustrating a fifth example of the marker according to the second embodiment;

FIG. 13 is a diagram illustrating a sixth example of the marker according to the second embodiment; and

FIG. 14 is a schematic configuration diagram of a computer according to the first and second embodiments.

### DETAILED DESCRIPTION OF EMBODIMENTS

Hereinafter, the present disclosure will be described through embodiments, but the disclosure according to the claims is not limited to the following embodiments. Moreover, not all of the configurations described in the embodiments are indispensable as means for solving the problem. For the sake of clarity, omission and simplification are made in the following description and drawings as appropriate. In the drawings, the same elements are designated by the same reference signs.

### First Embodiment

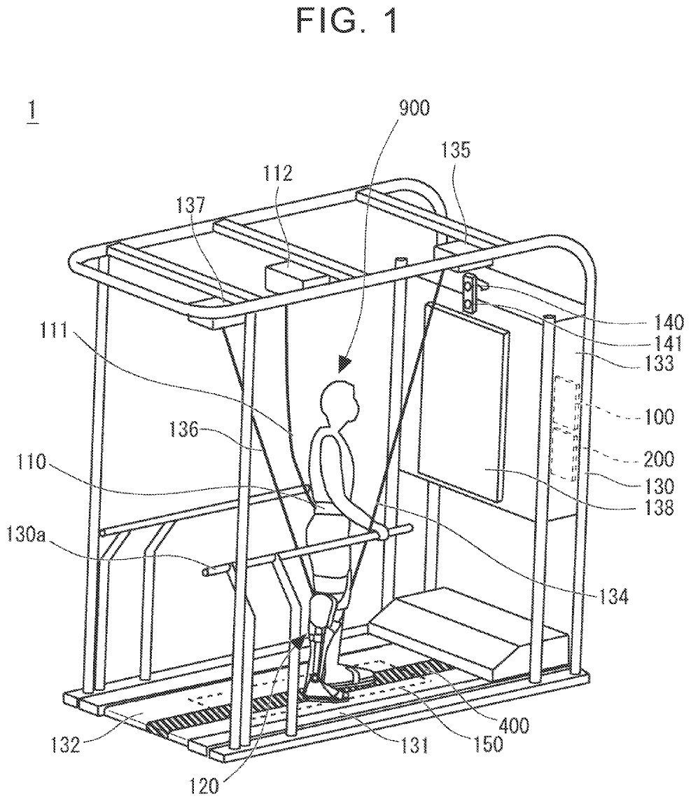

Hereinafter, a first embodiment of the present disclosure will be described. FIG. 1 is a schematic perspective view of a walking training system 1 according to the first embodiment. The walking training system 1 is an example of a walking state measurement device (also referred to as a walking state measurement system) according to the first embodiment. The walking training system 1 is a system for a trainee 900 who is a hemiplegic patient suffering from paralysis in one leg to perform walking training. The trainee 900 is also referred to as a subject. The up-down direction, the right-left direction, and the front-rear direction in the following description represent directions with the direction of the trainee 900 as a reference.

The walking training system 1 mainly includes a control panel 133 attached to a frame 130 constituting the entire skeleton, a treadmill 131 on which the trainee 900 walks, and a walking assist device 120 attached to an affected leg that is a leg of the trainee 900 on a paralyzed side.

The frame 130 is provided to stand on the treadmill 131 installed on the floor. The treadmill 131 rotates an endless belt 132 with a motor (not shown). Thus, the belt 132 travels along the orbit. The treadmill 131 is a device that prompts the trainee 900 to walk. The trainee 900 who performs walking training rides on the belt 132 and attempts a walking motion with respect to the walking surface provided on the belt 132 that travels in a walking front-rear direction. The belt 132 is provided with a marker 400, which will be described later.

The frame 130 supports a control panel 133 and a training monitor 138. The control panel 133 accommodates an information processing device 100 and a system control unit 200. The information processing device 100 is a computer device that corrects angle deviations and position deviations of a first camera 140 and a second camera 141, which will be described later. The system control unit 200 is a computer device that is connected to the information processing device 100 and a load distribution sensor 150 and controls various motors and sensors based on the information output from the information processing device 100 and the load distribution sensor 150.

The training monitor 138 is a display device that presents information on training and measurement to the trainee 900. The training monitor 138 is, for example, a liquid crystal panel. The training monitor 138 is installed such that the trainee 900 can visually recognize the training monitor 138 while walking on the belt 132 of the treadmill 131.

Further, the frame 130 supports a front tension unit 135 at the front of the overhead portion of the trainee 900, a harness tension unit 112 at the overhead portion, and a rear tension unit 137 at the rear of the overhead portion. The frame 130 may include handrails 130a for the trainee 900 to grab.

The first camera 140 is a camera unit for taking an image of the trainee 900 at such an angle of view that the gait of the trainee 900 can be recognized from the front. The first camera 140 includes a lens and an imaging element. The imaging element is, for example, a complementary metal oxide semiconductor (CMOS) image sensor, and converts an optical image on an image plane into an image signal. In the first embodiment, the first camera 140 is also referred to as red-green-blue (RGB) camera, and is installed above so as to provide such an angle of view that the whole body including the head of the trainee 900 standing on the belt 132 can be captured.

The second camera 141 is also a camera unit for taking an image of the trainee 900 at such an angle of view that the gait of the trainee 900 can be recognized from the front. The second camera 141 also includes a lens and an imaging element. In the first embodiment, the second camera 141 is also referred to as a depth camera. The second camera 141 may be a depth camera of a time of flight (TOF) method that measures a distance by measuring, for example, time from irradiation of a subject with light to reception of reflected light by an imaging element such as a CMOS image sensor. In the first embodiment, the second camera 141 is installed in the vicinity of the first camera 140, for example, at a position within a predetermined distance from the first camera 140. In this figure, the second camera 141 is installed below the first camera 140. In the first embodiment, the second camera 141 is also installed above so as to provide such an angle of view that the whole body including the head of the trainee 900 standing on the belt 132 can be captured.

Hereinafter, the first camera 140 and the second camera 141 may be collectively referred to as simply cameras.

The walking training system 1 may include a side camera unit that takes an image of the trainee 900 at such an angle of view that the gait of the trainee 900 can be recognized from the side. In this case, the side camera unit may be installed on the handrail 130a so as to capture the trainee 900 from the side.

One end of a front wire 134 is connected to a winding mechanism of the front tension unit 135, and the other end is connected to the walking assist device 120. The winding mechanism of the front tension unit 135 winds and unwinds the front wire 134 in accordance with the movement of the affected leg, by turning on and off a motor (not shown) following the instruction of the system control unit 200. Similarly, one end of a rear wire 136 is connected to a winding mechanism of the rear tension unit 137, and the other end is connected to the walking assist device 120. The winding mechanism of the rear tension unit 137 winds and unwinds the rear wire 136 in accordance with the movement of the affected leg, by turning on and off a motor (not shown) following the instruction of the system control unit 200. With such a coordinated operation of the front tension unit 135 and the rear tension unit 137, the load of the walking assist device 120 is offset so as not to be a burden on the affected leg, and further, the forward swing motion of the affected leg is assisted in accordance with the degree of the setting.

An operator (not shown) who is a training assistant sets the assist level high, for a trainee who has severe paralysis. The operator is a physiotherapist or a doctor who has the authority to select, correct, and add the setting items of the walking training system 1. When the assist level is set high, the front tension unit 135 winds up the front wire 134 with a relatively large force in accordance with the forward swing timing of the affected leg. As the training progresses and assistance becomes no longer needed, the operator sets the assist level to the minimum. When the assist level is set to the minimum, the front tension unit 135 winds up the front wire 134 with a force to cancel the weight of the walking assist device 120 in accordance with the forward swing timing of the affected leg.

The walking training system 1 includes a safety device including, as main components, a safety brace 110, a harness wire 111, and the harness tension unit 112. The safety brace 110 is a belt wrapped around the abdomen of the trainee 900 and is fixed to the waist by, for example, a hook-and-loop fastener. One end of the harness wire 111 is connected to the safety brace 110, and the other end is connected to the winding mechanism of the harness tension unit 112. The winding mechanism of the harness tension unit 112 winds and unwinds the harness wire 111 by turning on and off a motor (not shown). With such a configuration, when the trainee 900 significantly loses his/her posture, the safety device winds up the harness wire 111 following the instruction of the system control unit 200 that has detected the movement, and supports the upper body of the trainee 900 with the safety brace 110.

The walking assist device 120 is attached to the affected leg of the trainee 900 and assists the trainee 900 in walking by reducing the load of extension and bending on the knee joint of the affected leg. The walking assist device 120 transmits data on the leg movement acquired through the walking training to the system control unit 200, and drives the joint portion following the instruction from the system control unit 200. The walking assist device 120 can also be connected, via a wire or the like, to a hip joint (a connecting member including a rotating portion) attached to the safety brace 110 that is a part of the fall prevention harness device.

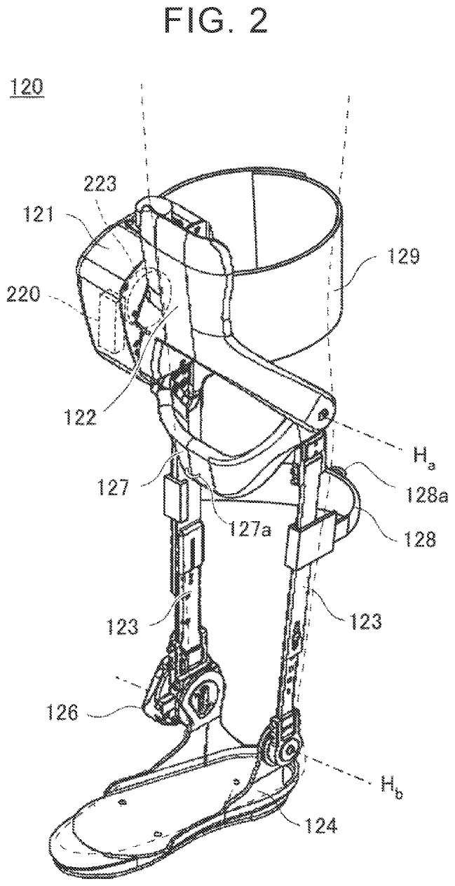

FIG. 2 is a schematic perspective view showing a configuration example of the walking assist device 120. The walking assist device 120 mainly includes a control unit 121 and a plurality of frames that supports various parts of the affected leg. The walking assist device 120 is also referred to as a leg robot.

The control unit 121 includes an auxiliary control unit 220 that controls the walking assist device 120, and also includes a motor (not shown) that generates a driving force for assisting the extension motion and the bending motion of the knee joint. The frames that support various parts of the affected leg include an upper leg frame 122 and lower leg frames 123 that are pivotably connected to the upper leg frame 122. The frames further include a foot flat frame 124 pivotably connected to the lower leg frames 123, a front connecting frame 127 for connecting the front wire 134, and a rear connecting frame 128 for connecting the rear wire 136.

The upper leg frame 122 and the lower leg frames 123 pivot relative to each other around a hinge axis Hₐ shown in the figure. The motor of the control unit 121 rotates following the instruction of the auxiliary control unit 220 to force the upper leg frame 122 and the lower leg frames 123 to relatively open and close around the hinge axis Hₐ. An angle sensor 223 accommodated in the control unit 121 is, for example, a rotary encoder, and detects the angle between the upper leg frame 122 and the lower leg frames 123 around the hinge axis Hₐ. The lower leg frames 123 and the foot flat frame 124 pivot relative to each other around a hinge axis H_{b} shown in the figure. The relative pivot angle range is adjusted in advance by an adjusting mechanism 126.

The front connecting frame 127 is provided so as to extend in the right-left direction on the front side of the upper leg and connect to the upper leg frame 122 at both ends. The front connecting frame 127 is further provided with a connecting hook 127a for connecting the front wire 134, around the center in the right-left direction. The rear connecting frame 128 is provided so as to extend in the right-left direction on the rear side of the lower leg and connect to the lower leg frames 123 at both ends. Further, the rear connecting frame 128 is provided with a connecting hook 128a for connecting the rear wire 136, around the center in the right-left direction.

The upper leg frame 122 is provided with an upper leg belt 129. The upper leg belt 129 is a belt integrally provided on the upper leg frame, and is wrapped around the upper leg portion of the affected leg to fix the upper leg frame 122 to the upper leg portion. This suppresses the entire walking assist device 120 from shifting with respect to the legs of the trainee 900.

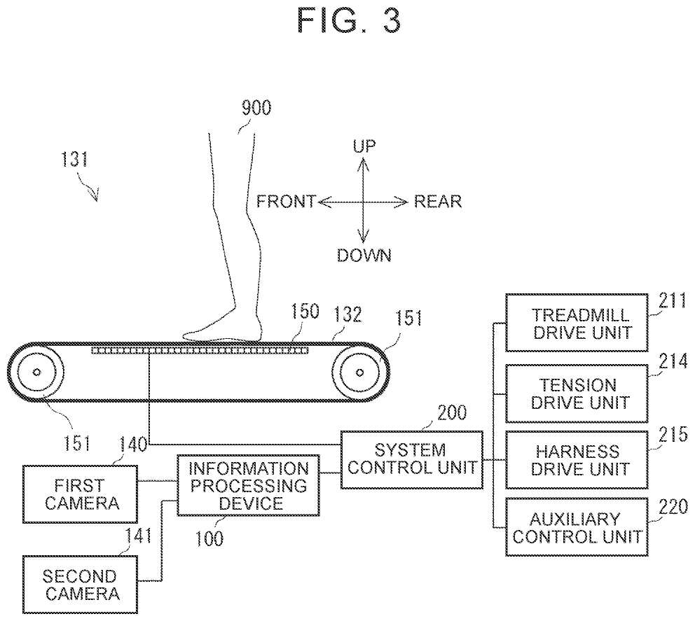

FIG. 3 is a diagram illustrating the operation of the system control unit according to the first embodiment. The treadmill 131 shown in the figure includes at least the ring-shaped belt 132, a pulley 151, and the motor (not shown). The belt 132 travels as the pulley 151 is rotated by the motor.

The load distribution sensor 150 is disposed inside the belt 132, that is, on the side of the belt 132 opposite from the surface on which the trainee 900 rides. The load distribution sensor 150 is fixed to the body of the treadmill 131 so as not to move together with the belt 132.

The load distribution sensor 150 is a load distribution sensor sheet with a plurality of pressure detection points. The pressure detection points are arranged in a matrix so as to be parallel with a walking surface (mounting surface) that supports the sole of the trainee 900 in the standing state. Further, the load distribution sensor 150 is disposed toward the center of the walking surface in the right-left direction that is orthogonal to the walking front-rear direction. The walking front-rear direction is a direction parallel to the traveling direction of the belt 132. By using the measurement results at the pressure detection points, the load distribution sensor 150 can detect the magnitude and the distribution of the vertical load received from the sole of the trainee 900. Thereby, the load distribution sensor 150 can detect the position of the sole of the trainee 900 in the standing state and the load received from the sole of the trainee 900, via the belt 132.

Here, the load distribution sensor 150 is connected to the system control unit 200, and outputs load distribution information as the measurement information to the system control unit 200.

The information processing device 100 is connected to the first camera 140 and the second camera 141. The information processing device 100 acquires an image from each of the first camera 140 and the second camera 141, and corrects at least one of the images using the camera parameters. The information processing device 100 is also connected to the system control unit 200, and supplies the images, at least one of which has been corrected, to the system control unit 200. The camera parameters are calculated in advance by the information processing device 100 based on the position of the marker 400 in each image.

The system control unit 200 measures the walking state of the trainee 900 based on the load distribution information output from the load distribution sensor 150 and the corrected image supplied from the information processing device 100. Specifically, the system control unit 200 uses the above-mentioned two types of images that are acquired from the information processing device 100 and at least one of which has been corrected, that is, calibrated, to generate three-dimensional position information of the body of the trainee 900. Then, the system control unit 200 detects the walking state of the trainee 900 based on the load distribution information and the three-dimensional position information.

The system control unit 200 controls various drive units based on the detected walking state. For example, the system control unit 200 is connected to a treadmill drive unit 211, a tension drive unit 214, a harness drive unit 215, and the auxiliary control unit 220 of the walking assist device 120 by wire or wirelessly. The system control unit 200 causes the treadmill drive unit 211, the tension drive unit 214, and the harness drive unit 215 to drive, and transmits a control signal to the auxiliary control unit 220.

The treadmill drive unit 211 includes the above motor for rotating the belt 132 of the treadmill 131 and a drive circuit thereof. The system control unit 200 performs rotation control of the belt 132 by transmitting a drive signal to the treadmill drive unit 211. The system control unit 200 adjusts the rotation speed of the belt 132 in accordance with, for example, the walking speed set by the operator. Alternatively, the system control unit 200 adjusts the rotation speed of the belt 132 in accordance with the walking state acquired from the information processing device 100.

The tension drive unit 214 includes a motor for tensioning the front wire 134 and a drive circuit thereof that are provided in the front tension unit 135, and a motor for tensioning the rear wire 136 and a drive circuit thereof that are provided in the rear tension unit 137. The system control unit 200 controls the winding of the front wire 134 and the winding of the rear wire 136 by transmitting a drive signal to the tension drive unit 214. Further, the system control unit 200 controls the tensile force of each wire by controlling the driving torque of the motor as well as the winding operation. Further, the system control unit 200 identifies the timing at which the affected leg switches from the standing state to the swinging state based on the walking state of the trainee 900 output from the information processing device 100, and increases or decreases the tensile force of each wire in synchronization with that timing, thereby assisting the motion of the affected leg.

The harness drive unit 215 includes a motor for tensioning the harness wire 111 and a drive circuit thereof that are provided in the harness tension unit 112. The system control unit 200 controls the winding of the harness wire 111 and the tensile force of the harness wire 111 by transmitting a drive signal to the harness drive unit 215. For example, when the trainee 900 is predicted to fall, the system control unit 200 winds up the harness wire 111 by a certain amount to suppress the trainee from falling.

The auxiliary control unit 220 is, for example, a microprocessor unit (MPU), and performs control of the walking assist device 120 by executing a control program provided from the system control unit 200. Moreover, the auxiliary control unit 220 notifies the system control unit 200 of the state of the walking assist device 120. Further, the auxiliary control unit 220 receives a command from the system control unit 200 and performs control of starting, stopping, and the like of the walking assist device 120.

The auxiliary control unit 220 transmits a drive signal to the joint drive unit including the motor of the control unit 121 and the drive circuit thereof, to force the upper leg frame 122 and the lower leg frames 123 to relatively open and close around the hinge axis Hₐ. Such motions assist the extension motion and the bending motion of the knee and suppress knee collapse. The auxiliary control unit 220 receives a detection signal from an angle sensor (not shown) that detects the angle between the upper leg frame 122 and the lower leg frames 123 around the hinge axis Hₐ, and calculates the opening angle of the knee joint.

Here, the issue of the present embodiment will be described again with reference to FIG. 1. For example, if a marker used for calculating camera parameters is provided on a three-dimensional object (sometimes called a booth frame) of the frame 130, the information processing device 100 tends to erroneously recognize the marker due to the influence of parallax. Therefore, it is desirable that the marker be installed on a flat surface portion. When the marker is provided on flat surface portions of the treadmill 131 on both sides of the frame 130, the markers are located at the ends of the field of view of the camera. Therefore, the markers are easily affected by the distortion of the peripheral portion of the lens. Thus, the information processing device 100 tends to erroneously recognize the marker.

In the walking training system 1, a marker 400 is provided on the belt 132 of the treadmill 131 along the circumferential direction of the belt 132. The marker 400 is provided so as to be present on at least the walking surface (that is, the surface on the trainee 900 side) of the belt 132 even when the belt 132 rotates. With this, even when the belt 132 rotates, the marker is always present on the walking surface, so that the camera can easily capture the marker 400. Further, when the marker 400 is provided on the belt 132 of the treadmill 131, the marker 400 is located toward the center of the field of view of the camera installed in front, as compared with the case where the marker 400 is provided on the flat surface portions of the treadmill 131 on both sides. Thus, it is possible to reduce the influence of distortion in the peripheral portion of the lens.

In the first embodiment, the marker 400 is provided so as to be endless over the entire circumference of the belt 132. With this, the marker 400 is always present on the walking surface even when the belt 132 rotates. The width of the marker 400 may be constant. With this, even when the belt 132 rotates, the position and the shape of the marker 400 do not change, and erroneous recognition of the marker 400 can be suppressed.

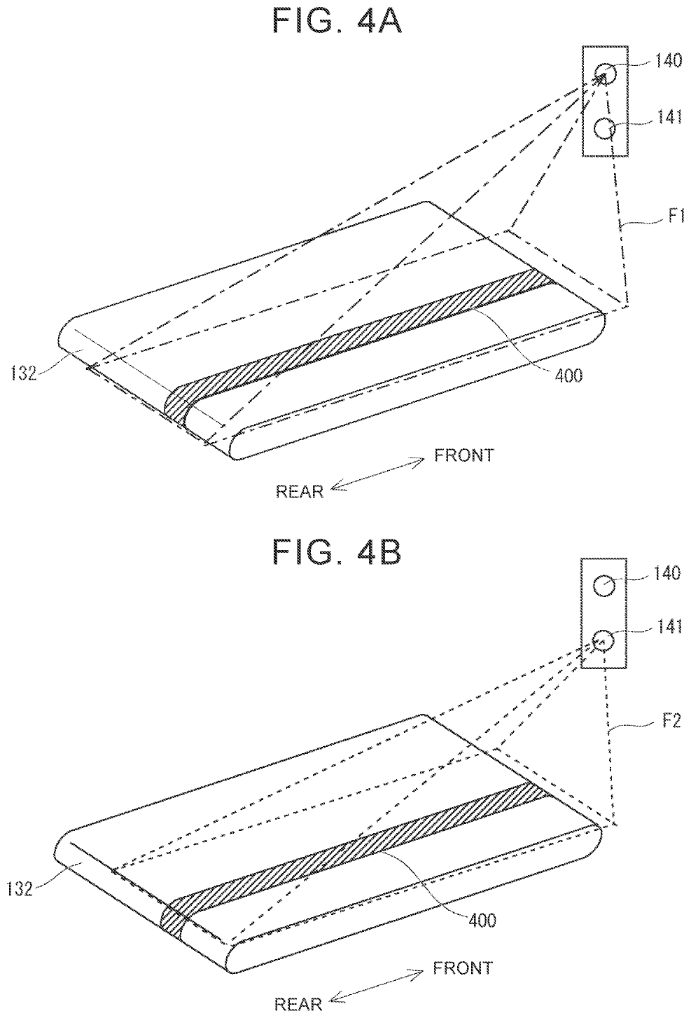

Next, the way in which the first camera 140 and the second camera 141 take images of the marker 400 will be described. FIGS. 4A and 4B are perspective views showing the belt 132 and the field of view of the camera according to the first embodiment. FIG. 4A shows a field of view F1 of the first camera 140, and FIG. 4B shows a field of view F2 of the second camera 141.

The first camera 140 takes an image of the belt 132 and the whole body of the trainee 900 (not shown) on the belt 132 from the front and above. Therefore, the field of view F1 of the first camera 140 includes the entire marker 400 on the walking surface of the belt 132. The same applies to the field of view F2 of the second camera 141. However, the second camera 141 is installed below the first camera 140, and the field of view F2 is located at a different position from the field of view F1. Further, the angle of view of the second camera 141 may be different from the angle of view of the first camera 140.

Here, the first camera 140 generates a first image 300 that is an image of the marker 400 on the walking surface taken from above. The second camera 141 generates a second image 320 that is an image of the marker 400 on the walking surface taken from above.

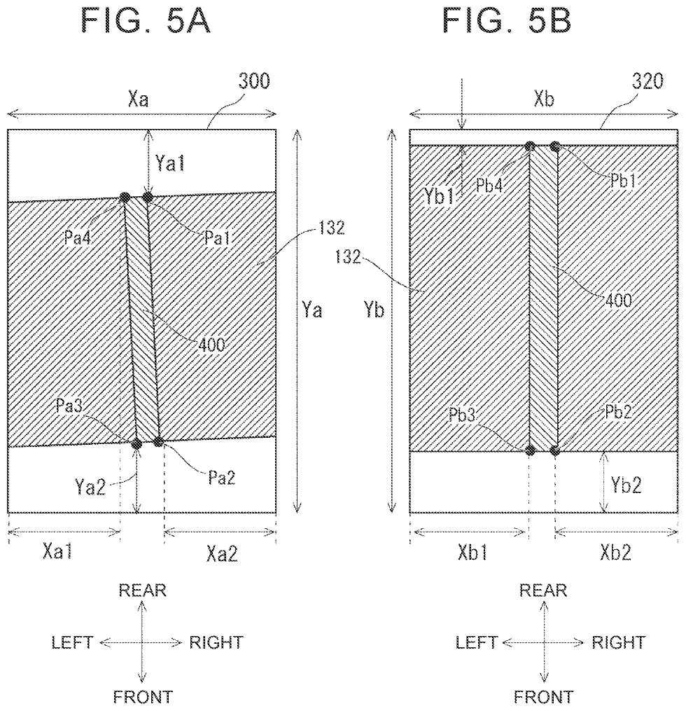

FIGS. 5A and 5B are diagrams showing an example of the first image 300 and the second image 320 according to the first embodiment. FIG. 5A shows the first image 300 and FIG. 5B shows the second image 320. The right-left direction in the figures corresponds to the walking right-left direction, and the up-down direction in the figures corresponds to the walking front-rear direction. The first image 300 and the second image 320 each include an image area of the marker 400. The hatched area in each figure is the image area of the belt 132 in the image.

As shown in FIG. 5A, the first image 300 has a pixel count of Xa×Ya. For example, the image area of the marker 400 in the first image 300 includes feature points Pa1 to Pa4. For example, the feature points are points extracted by edge processing, template matching, and the like. In the first image 300, the feature points Pa1 to Pa4 are intersections of the most end portions of the belt 132 (folding portions of the belt 132 and the boundaries defining the image area of the belt 132) and the marker 400.

The leftmost feature point in the image area of the marker 400 in the first image 300 is the feature point Pa4. The feature point Pa4 is located toward the center from the left end of the first image 300 by a pixel count of Xa1 (<Xa). Similarly, the rightmost feature point in the image area of the marker 400 in the first image 300 is the feature point Pa2. The feature point Pa2 is located toward the center from the right end of the first image 300 by a pixel count of Xa2 (<Xa). That is, for the first image 300, it can be said that the pixel count from the right end (or left end) of the first image 300 to the right end (or left end) of the marker 400 in the walking right-left direction is equal to or more than a predetermined threshold value. The threshold value is less than Xa and is determined based on the lens performance.

The uppermost feature point in the image area of the marker 400 in the first image 300 is the feature point Pa1. The feature point Pa1 is located toward the center from the upper end of the first image 300 by a pixel count of Ya1 (<Ya). The lowermost feature point in the image area of the marker 400 in the first image 300 is the feature point Pa3. The feature point Pa3 is located toward the center from the lower end of the first image 300 by a pixel count of Ya2 (<Ya). That is, for the first image 300, it can be said that the pixel count from the upper end (or lower end) of the first image 300 to the upper end (or lower end) of the marker 400 in the walking front-rear direction is equal to or more than a predetermined threshold value. The threshold value is less than Ya and is determined based on the lens performance.

On the other hand, as shown in FIG. 5B, the second image 320 has a pixel count of Xb×Yb. For example, the image area of the marker 400 in the second image 320 includes feature points Pb1 to Pb4. Also in the second image 320, the feature points Pa1 to Pa4 are the intersections of the most end portions of the belt 132 and the marker 400.

Similarly in this figure, for the second image 320, the pixel count from the right end (or left end) of the second image 320 to the right end (or left end) of the marker 400 in the walking right-left direction is Xb1, Xb2, and is equal to or more than a predetermined threshold value. The threshold value is less than Xb and is determined based on the lens performance. Also, for the second image 320, the pixel count from the upper end (or lower end) of the second image 320 to the upper end (or lower end) of the marker 400 in the walking front-rear direction is Yb1, Yb2 in this figure, and is equal to or more than a predetermined threshold value. The threshold value is less than Yb and is determined based on the lens performance.

By disposing the marker 400 toward the center of the field of view of the camera based on the lens performance in this way, it is possible to reduce the influence of distortion in the peripheral portion of the lens.

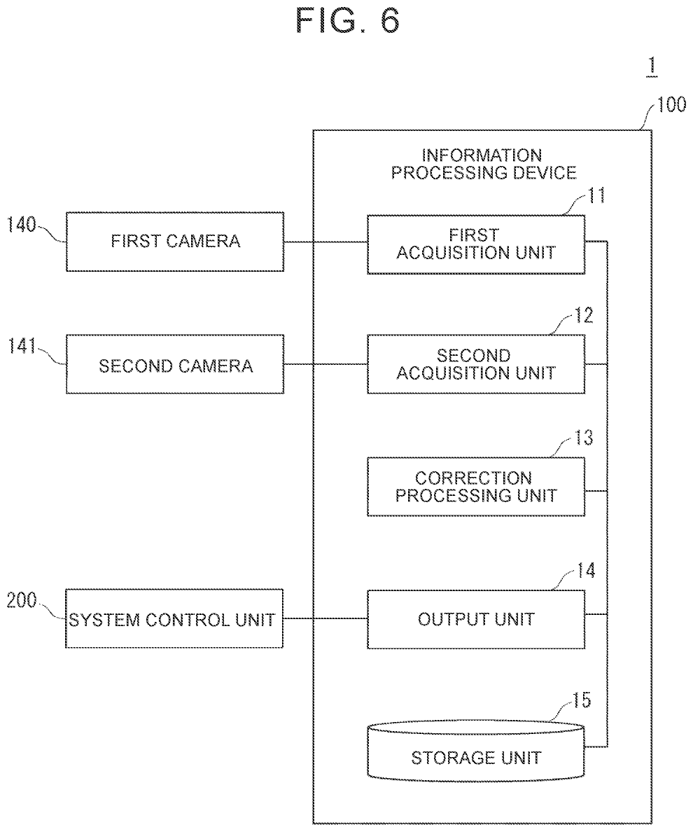

FIG. 6 is a block diagram showing a functional configuration of the information processing device 100 according to the first embodiment. The information processing device 100 includes a first acquisition unit 11, a second acquisition unit 12, a correction processing unit 13, an output unit 14, and a storage unit 15.

The first acquisition unit 11 is connected to the first camera 140 and acquires the first image 300 from the first camera 140 during camera calibration and walking training. The first acquisition unit 11 supplies the first image 300 to the correction processing unit 13.

The second acquisition unit 12 is connected to the second camera 141 and acquires the second image 320 from the second camera 141 during camera calibration and walking training. The second acquisition unit 12 supplies the second image 320 to the correction processing unit 13.

The correction processing unit 13 corrects at least one of the first image 300 and the second image 320 acquired during the walking training, based on the position of the image area of the marker 400 included in each of the first image 300 and the second image 320 acquired at the time of camera calibration. Specifically, the correction processing unit 13 calculates the correction parameters as the camera parameters based on the positions of the feature points in the image area of the marker 400 in the first image 300 acquired at the time of camera calibration and the positions of the corresponding feature points in the image area of the marker 400 in the second image 320 acquired at the time of camera calibration. For example, the correction parameters are parameters used for transformation for matching each of the feature points in the image area of the marker 400 in the first image 300 with a corresponding feature point in the image area of the marker 400 in the second image 320. Then, the correction processing unit 13 transforms at least one of the first image 300 and the second image 320 acquired during the walking training using the correction parameters. Linear transformation, for example, affine transformation, may be used for the transformation.

The correction processing unit 13 supplies the first image 300 and the second image 320, at least one of which has been corrected, to the output unit 14. It should be noted that “the first image 300 and the second image 320, at least one of which has been corrected” may be simply referred to as “the first image 300 and the second image 320 after correction processing”.

The output unit 14 is connected to the system control unit 200, and outputs (transmits) the first image 300 and the second image 320 after correction processing to the system control unit 200.

The storage unit 15 is a storage medium that stores information necessary for the correction processing of the information processing device 100, such as correction parameters and the like.

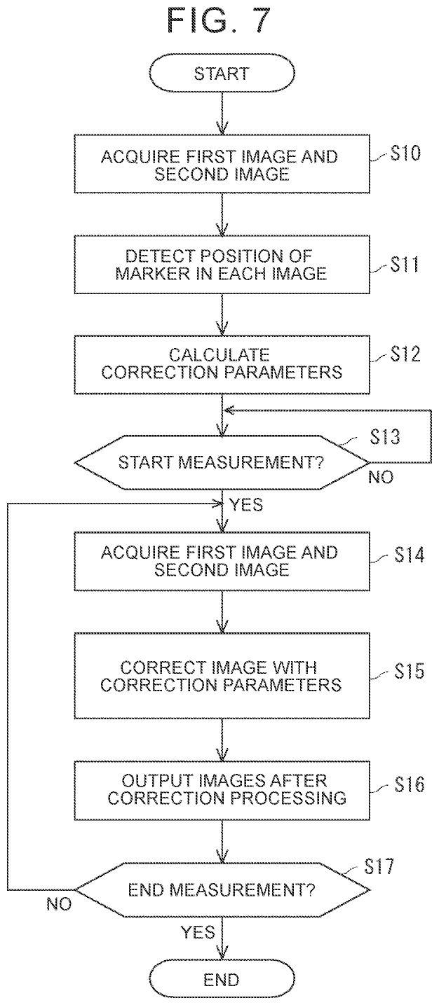

FIG. 7 is a flowchart showing an information processing procedure of the information processing device 100 according to the first embodiment.

First, the first acquisition unit 11 of the information processing device 100 acquires the data of the first image 300 from the first camera 140, and the second acquisition unit 12 acquires the second image 320 from the second camera 141 (step S10).

Next, the correction processing unit 13 detects the position of the marker 400 included in each image (first image 300, second image 320) (step S11). For example, the correction processing unit 13 detects three or more feature points of the marker 400 included in each image. Then, the correction processing unit 13 associates each feature point included in the first image 300 with each feature point included in the second image 320.

Then, the correction processing unit 13 calculates the correction parameters for matching each feature point in the first image 300 with a corresponding feature point in the second image 320 based on the position coordinates of the feature points associated with each other (step S12). For example, the correction processing unit 13 calculates affine transformation parameters as the correction parameters based on the position coordinates of the associated feature points, and stores the parameters in the storage unit 15. When correcting either the first image 300 or the second image 320 in step S16 described later, the correction processing unit 13 calculates the correction parameters for one type of image. However, when correcting both, the correction processing unit 13 calculates the correction parameters for each image.

Subsequently, the information processing device 100 determines whether to start measurement (step S13). For example, starting measurement includes a case where the operator has input start of measurement. When the information processing device 100 does not start the measurement (No in step S13), this process is repeated, and when the information processing device 100 starts the measurement (Yes in step S13), the process proceeds to step S14.

The first acquisition unit 11 and the second acquisition unit 12 acquire the first image 300 and the second image 320, respectively, in response to the start of measurement (step S14).

Then, the correction processing unit 13 corrects the first image 300 or the second image 320 using the calculated correction parameters (step S15), and outputs the images after correction processing (step S16). For example, the correction processing unit 13 corrects the first image 300 by affine transformation using the correction parameters. Then, the correction processing unit 13 associates the corrected first image 300 with the second image 320 acquired in step S14 and transmits the images to the system control unit 200. The correction processing unit 13 may correct both the first image 300 and the second image 320 using the correction parameters. In this case, the correction processing unit 13 associates the corrected first image 300 with the corrected second image 320 and transmits the images to the system control unit 200.

Then, the information processing device 100 determines whether to end the measurement (step S17). For example, ending the measurement includes a case where the operator has input ending of measurement. When the information processing device 100 does not end the measurement (No in step S17), the process returns to step S14, and when the information processing device 100 ends the measurement (Yes in step S17), the process ends.

In the above example, the correction processing unit 13 performs camera calibration before the starting of measurement, that is, calculates correction parameters. However, the correction processing unit 13 may calculate the correction parameters during the measurement instead of before the start of measurement, or may update the correction parameters during the measurement in addition to before the start of measurement. Then, the correction processing unit 13 corrects the image using the latest correction parameters. The marker 400 is provided so as to be always present on the walking surface even when the belt 132 rotates. Therefore, this process can be realized.

As described above, with the walking training system 1 of the first embodiment, the marker 400 is provided along the circumferential direction of the belt 132 so as to be present at least on the walking surface. Therefore, the information processing device 100 can detect the marker 400 from the captured image even when the belt 132 rotates. Further, the marker 400 is provided toward the center of the field of view of the camera. Thus, it is possible to reduce the influence of distortion in the peripheral portion of the lens. In this way, the walking training system 1 can suppress erroneous recognition of the marker 400 in calibration of a plurality of cameras. Thus, the walking training system 1 can improve the accuracy of determining the walking state, and can appropriately assist the joints.

### Second Embodiment

Next, a second embodiment will be described. In the first embodiment, the intersections between the most end portions of the belt 132 and the marker 400 are extracted as the feature points on the image. However, since the most end portions of the belt 132 on the image are folding portions, the shape of the marker 400 is likely to be distorted and the most end portions are likely to be located at the end portions of the field of view of the camera. Further, when the camera cannot sufficiently capture the most end portions of the belt 132 due to the position deviation of the camera, it is difficult to calibrate the camera correctly. Thus, in order to suppress such a situation, it is required to extract the feature points of the marker 400 while avoiding the most end portions of the belt 132. In the second embodiment, the marker is composed of a plurality of individual markers arranged at intervals of a predetermined distance or more over the entire circumference of the belt 132. This enables the features points of the markers to provide a plurality of feature points inward of the most end portion of the belt, that is, on the inner side of the walking surface. The correction processing unit 13 of the information processing device 100 can easily extract the feature points of the markers while avoiding the most end portions of the belt 132. Thus, even when one of the cameras cannot sufficiently capture the most end portions of the belt, the feature points of the markers can be detected. In addition, the influence of distortion in the peripheral portion of the lens can be reduced.

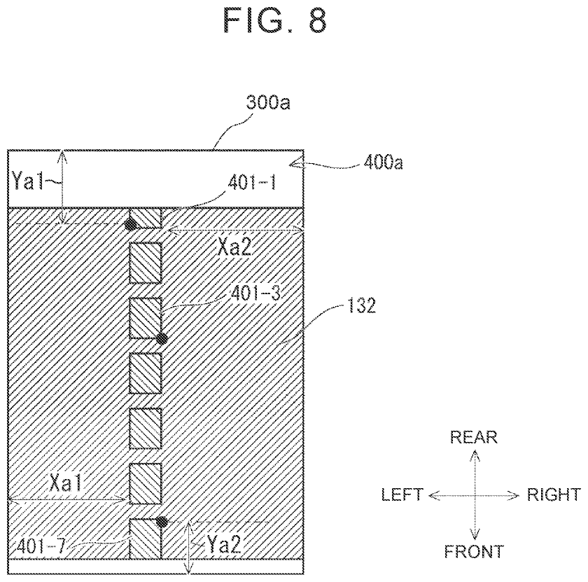

FIG. 8 is a diagram illustrating a first example of the marker according to the second embodiment. In this figure, a first image 300a including an image area of a marker 400a is shown.

The marker 400a includes a plurality of individual markers 401-1, 2, . . . 7 in order from the rearmost in the walking direction. The number of individual markers 401 is not limited to seven. The individual markers 401 have the same shape, and in this figure, each individual marker 401 has a rectangular shape with the same length and width. Each individual marker 401 is disposed spaced apart from the adjacent individual marker 401 by a predetermined distance or more along the circumferential direction of the belt 132. In this figure, the interval between the adjacent individual markers 401 is constant, but is not limited to this. The shape of the image area of the individual marker 401-1 in the first image 300a is different from the shapes of the image areas of the other individual markers 401 because the individual marker 401-1 is located at the folding portion of the belt 132.

For example, the correction processing unit 13 of the information processing device 100 extracts, as the feature points, a lower left end of the individual marker 401-1 located at the top, a lower right end of the third individual marker 401-3 from the top, and an upper right end of the seventh individual marker 401-7 from the top, in the first image 300a.

For the first image 300a as well, it can be said that the pixel counts from both ends of the first image 300a to both ends of the marker 400a in the walking right-left direction are equal to or more than a predetermined threshold value. The threshold value is less than Xa and is determined based on the lens performance.

For the first image 300a, the pixel count from the right end (or left end) of the first image 300a to the right end (or left end) of at least one individual marker 401 in the walking right-left direction is Xa1, Xa2, and is equal to or more than a predetermined threshold value. For the first image 300a, the pixel count from the upper end (or lower end) of the first image 300a to the upper end (or lower end) of at least one individual marker 401 in the walking front-rear direction is Ya1, Ya2, and is equal to or more than a predetermined threshold value.

Similarly, the correction processing unit 13 extracts, as the feature points, a lower left end of the individual marker located at the top, a lower right end of the third individual marker from the top, and an upper right end of the seventh individual marker from the top, in the second image 320a. Similarly, for the second image 320a, the pixel count from the right end (or left end) of the second image 320a to the right end (or left end) of at least one individual marker 401 in the walking right-left direction is equal to or more than a predetermined threshold value. The pixel count from the upper end (or lower end) of the second image 320a in the walking front-rear direction to the upper end (or lower end) of at least one individual marker 401 is equal to or more than a predetermined threshold value.

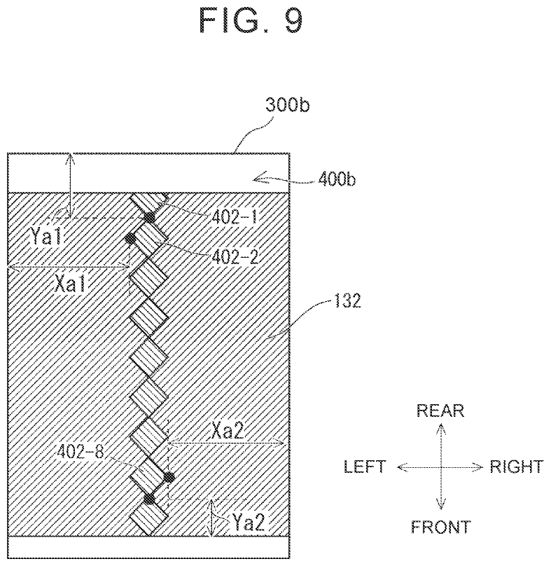

FIG. 9 is a diagram illustrating a second example of the marker according to the second embodiment. In this figure, a first image 300b including an image area of a marker 400b is shown.

The marker 400b includes a plurality of individual markers 402-1, 2, . . . 9 in order from the rearmost in the walking direction. The number of individual markers 402 is not limited to nine. The individual markers 402 have the same shape, and in this figure, each individual marker 402 has a rhombus shape with the same length and width. The individual markers 402 are arranged in such a manner that each individual marker 402 shares an upper or lower end point with an adjacent individual marker 402.

For example, the correction processing unit 13 of the information processing device 100 extracts, as the feature points, a lower end point (connection point with respect to the individual marker 402-2) of the individual marker 402-1 located at the top, a left end point of the second individual marker 402-2 from the top, and a lower end point (connecting point with respect to the individual marker 402-9) and a right end point of the eighth individual marker 402-8 from the top, in the first image 300b. The same applies to the second image 320b (not shown).

The pixel count from the upper end (or lower end) of the first image 300b to the upper end (or lower end) of the marker 400b and the pixel count from the right end (or left end) of the first image 300b to the right end (or left end) of the marker 400b are similar to those of the first image 300a.

In the first and second examples, the case where the individual markers have the same shape has been described. However, when all the individual markers have the same shape, for example, the lower end point of the individual marker 402-8 is recognized as a feature point in the first image 300b, and the individual marker 402-7 that is different from the individual marker 402-8 may be recognized as a feature point corresponding to the above feature point in the second image 320b. This causes a problem that the calculation accuracy of the correction parameters is lowered due to the erroneous recognition of the correspondence of the feature points and the calibration between the cameras is not performed correctly. To suppress this, the markers may be composed of individual markers having different shapes or colors from each other, at least on the walking surface.

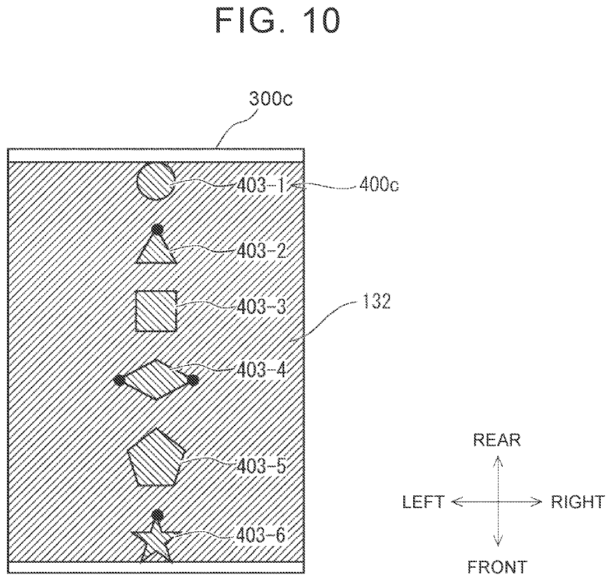

FIG. 10 is a diagram illustrating a third example of the marker according to the second embodiment. In this figure, a first image 300c including an image area of a marker 400c is shown.

The marker 400c includes a plurality of individual markers 403-1, 2, . . . 6 in order from the rearmost in the walking direction. The number of individual markers 403 is not limited to six. The individual markers 403 have different shapes from each other. In this figure, the shapes of the individual markers 403-1, 2, . . . 6 are a circle, a triangle, a rectangle, a rhombus, a pentagon, and a star. Each individual marker 403 is separated from the adjacent individual marker 403 at a predetermined distance or more.

For example, the correction processing unit 13 of the information processing device 100 extracts, as the feature points, the upper end point of the triangular individual marker 403-2, the left and right end points of the rhombus-shaped individual marker 403-4, and the upper end point of the star-shaped individual marker 403-6 in the first image 300c. The same applies to the second image 320c (not shown). The correction processing unit 13 of the information processing device 100 calculates the correction parameters by associating with each other the feature points of the individual markers 403 recognized as having the same shape.

The individual markers 403 on the surface of the belt 132 that is not present as the walking surface (that is, the surface traveling in the direction opposite to the direction in which the walking surface travels) also have different shapes from each other, and the shapes may be different from those of the individual markers 403-1, 2, . . . 6. That is, the markers may be composed of individual markers having different shapes (or colors) from each other over the entire circumference of the belt 132.

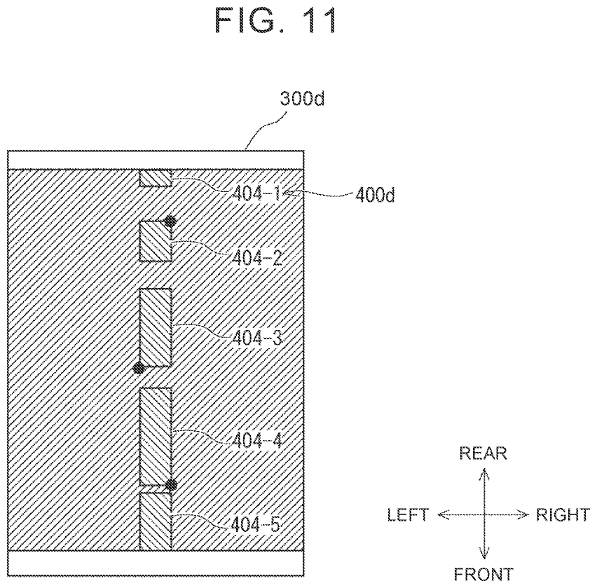

FIG. 11 is a diagram illustrating a fourth example of the marker according to the second embodiment. In this figure, a first image 300d including an image area of a marker 400d is shown.

The marker 400d includes a plurality of individual markers 404-1, 2, . . . 5 in order from the rearmost in the walking direction. The number of individual markers 404 is not limited to five. The individual markers 404 have rectangular shapes with the same lengths in the width direction but different aspect ratios. Each individual marker 404 is separated from the adjacent individual marker 404 at a predetermined interval.

For example, the correction processing unit 13 of the information processing device 100 extracts, as the feature points, an upper right end point of the individual marker 404-2 having an aspect ratio of a first value, a lower left end point of the individual marker 403-3 having an aspect ratio of a second value, and a lower right end point of the individual marker 404-4 having an aspect ratio of a third value in the first image 300d. The same applies to the second image. The correction processing unit 13 of the information processing device 100 calculates the correction parameters by associating with each other the feature points of the individual markers 404 recognized as having the same shape.

The individual markers 404 on the surface of the belt 132 that does is not present as the walking surface (that is, the surface traveling in the direction opposite to the direction in which the walking surface travels) also have different aspect ratios from each other, and the aspect ratios may be different from those of the individual markers 404-1, 2, . . . 5. That is, the markers may be composed of individual markers having different aspect ratios from each other over the entire circumference of the belt 132.

In the third to fourth examples, it is assumed that the markers are composed of individual markers having different shapes, colors or aspect ratios from each other on the walking surface. However, the markers may also include individual markers having the same shape, color or aspect ratio on the walking surface.

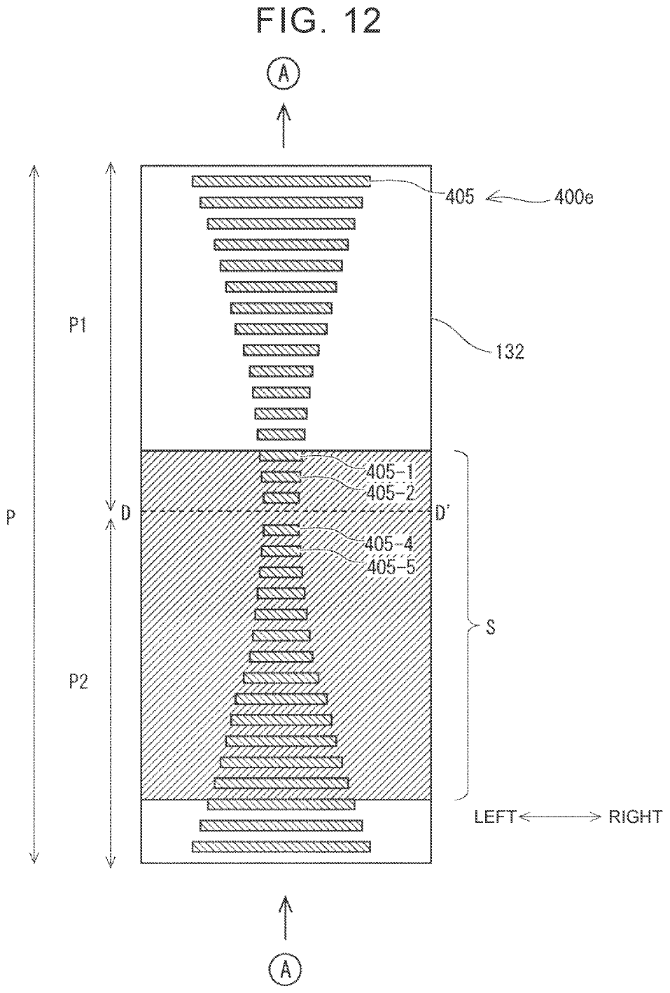

FIG. 12 is a diagram illustrating a fifth example of the marker according to the second embodiment. This figure shows a developed view of the belt 132. The right-ascending hatching area in this figure indicates the walking surface S at a certain point in time.

The belt 132 is provided with a pattern of individual markers 405 having a half circumference (P1, P2) of the belt 132 as a half cycle and the entire circumference (P) as one cycle. Each individual marker 405 on each half circumference P1, P2 is separated from the adjacent individual marker 405 at a predetermined interval. The individual markers 405 on each half circumference P1, P2 have the same length in the walking front-rear direction, but the lengths in the width direction are different in a stepwise manner. Therefore, the individual markers 405 on each half circumference P1, P2 have aspect ratios that are different in a stepwise manner. The individual markers 405 on the half circumferences P1 and P2 are line-symmetrical with respect to a boundary line D-D′. Therefore, when the walking surface S crosses the boundary line D-D′, there are two individual markers 405 having the same shape on the walking surface S.

However, even in such a case, the correction processing unit 13 of the information processing device 100 can identify the corresponding individual markers 405 in the images (first and second images), based on the rate of change or the value of difference between the aspect ratio of the individual marker 405 to be extracted as a feature point and the aspect ratio of the individual marker 405 adjacent thereto. For example, the second individual marker 405-2 from the top on the walking surface S has the same aspect ratio as the fifth individual marker 405-5 from the top. However, the aspect ratio of the individual marker 405-2 is larger than that of the first individual marker 405-1 from the top. In contrast, the aspect ratio of the individual marker 405-5 is smaller than that of the fourth individual marker 405-4 from the top. Therefore, the correction processing unit 13 of the information processing device 100 can distinguish the individual marker 405-2 from the individual marker 405-5.

The markers may have a pattern in which the half circumference of the belt 132 is one cycle, and individual markers having the same shape may be included in one pattern.

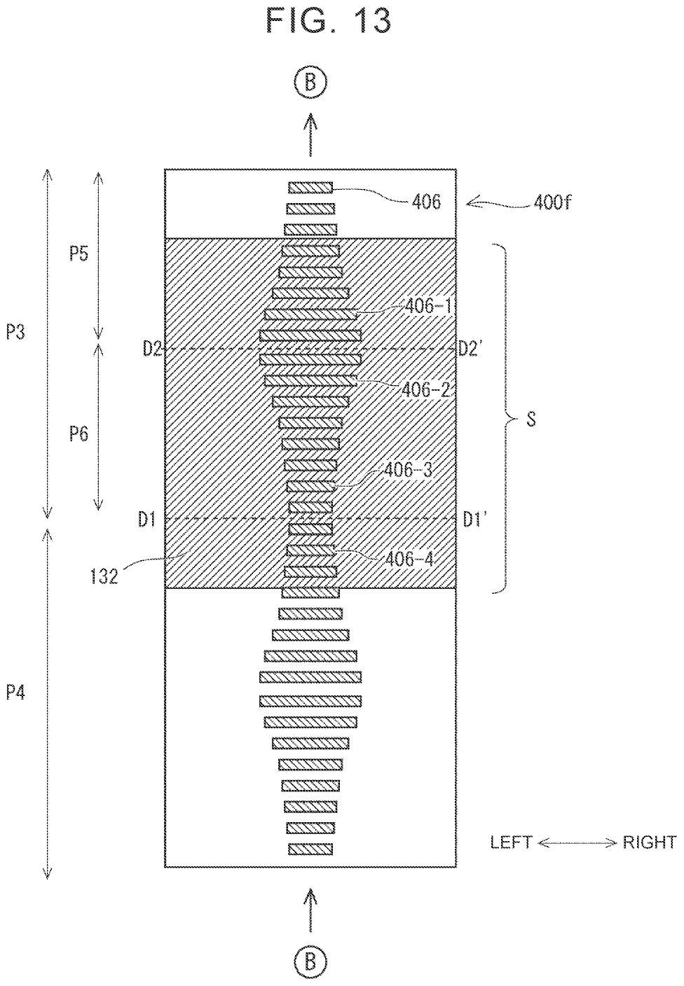

FIG. 13 is a diagram illustrating a sixth example of the marker according to the second embodiment. This figure shows a developed view of the belt 132. The right-ascending hatching area in this figure indicates the walking surface S at a certain point in time.

The belt 132 is provided with a pattern of individual markers 406 having a half circumference (P3, P4) of the belt 132 as one cycle. Each individual marker 406 on each half circumference P3, P4 is separated from the adjacent individual marker 406 at a predetermined interval. The half circumference P3 and the half circumference P4 are defined by a boundary line D1-D1′. Therefore, when the walking surface S crosses the boundary line D1-D1′, there are two or more individual markers 406 having the same shape on the walking surface S.

The individual markers 406 on quarter circumferences P5, P6 included in the half circumference P3 are arranged so as to be line-symmetrical with respect to a boundary line D2-D2′. The individual markers 406 on each quarter circumference P5, P6 have the same length in the walking front-rear direction, but the lengths in the width direction are different in a stepwise manner. Therefore, the individual markers 406 on each quarter circumference P5, P6 have aspect ratios that are different in a stepwise manner. Therefore, when the walking surface S crosses the boundary line D2-D2′, there are two individual markers 406 having the same shape on the walking surface S.

However, even in such a case, the correction processing unit 13 of the information processing device 100 can identify the corresponding individual markers 406 in the images (first and second images), based on the rate of change or the value of difference between the aspect ratio of the individual marker 406 to be extracted as a feature point and the aspect ratio of the individual marker 406 adjacent thereto, similarly to the fifth example. For example, the fourth individual marker 406-1 from the top on the walking surface S has the same aspect ratio as the seventh individual marker 406-2 from the top. There is the boundary line D2-D2′ between the two individual markers. However, the correction processing unit 13 of the information processing device 100 can distinguish the individual marker 406-1 having the aspect ratio smaller than that of the individual marker 406 located on the upper side thereof and adjacent thereto, from the individual marker 406-2 having the aspect ratio larger than that of the individual marker 406 located on the upper side thereof and adjacent thereto. This also applies to the individual marker 406-3 and the individual marker 406-4 with the boundary line D1-D1′ therebetween.

In the above-described embodiments, the present disclosure has been described as a hardware configuration, but the present disclosure is not limited thereto. The present disclosure can also be realized, for example, by causing a processor of a computer to execute a computer program regarding various processes.

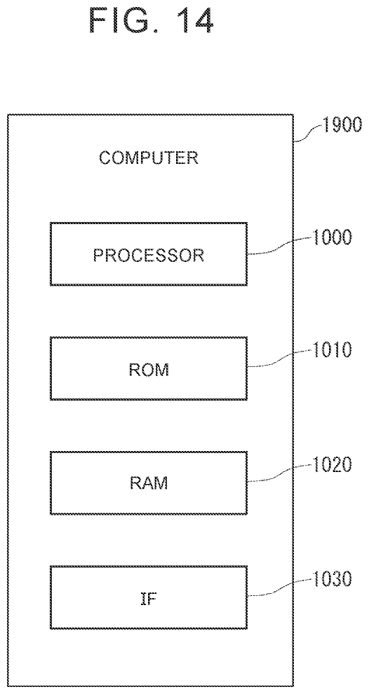

FIG. 14 is a schematic configuration diagram of a computer 1900 according to the first and second embodiments.

The computer 1900 includes a processor 1000, a read only memory (ROM) 1010, a random access memory (RAM) 1020, and an interface unit 1030 (IF; interface) as main hardware configurations. The processor 1000, the ROM 1010, the RAM 1020 and the interface unit 1030 are connected to each other via a data bus and the like.

The processor 1000 has a function as an arithmetic device that performs a control process, an arithmetic process, and the like. The processor 1000 may be a central processing unit (CPU), a graphics processing unit (GPU), a field-programmable gate array (FPGA), a digital signal processor (DSP), an application specific integrated circuit (ASIC), or a combination thereof. The ROM 1010 has a function for storing a control program, an arithmetic program, and the like executed by the processor 1000. The RAM 1020 has a function for temporarily storing processing data and the like. The interface unit 1030 inputs and outputs signals to and from the outside by wire or wirelessly. The interface unit 1030 receives the operation of inputting data by the user and displays information to the user. For example, the interface unit 1030 communicates with the first camera 140 and the second camera 141.

In the examples described above, the program can be stored using various types of non-transitory computer-readable medium as an example of the ROM 1010 and supplied to the computer 1900. The non-transitory computer-readable medium include various types of tangible storage medium. Examples of the non-transitory computer-readable medium include magnetic recording medium (for example, flexible disks, magnetic tapes, hard disk drives), magneto-optical recording medium (for example, magneto-optical disks), compact disc read-only memory (CD-ROM), compact disc recordable (CD-R), compact disc rewritable (CD-R/W), and a semiconductor memory (for example, mask ROM, programmable ROM (PROM), erasable PROM (EPROM), flash ROM, random access memory (RAM)). The program may also be supplied to the computer by various types of transitory computer-readable medium. Examples of the transitory computer-readable medium include electrical signals, optical signals, and electromagnetic waves. The transitory computer-readable medium can supply a program to the computer 1900 via a wired communication path such as an electric wire and an optical fiber, or a wireless communication path.

In the above embodiments, the computer 1900 is composed of a computer system including a personal computer, a word processor, and the like. However, the present disclosure is not limited thereto, and the computer 1900 can be composed of a local area network (LAN) server, a host computer of computer (personal computer) communication, a computer system connected on the Internet, and the like. The computer 1900 can be composed of the network as a whole with the functions being distributed to various devices on the network. Thus, the components of the information processing device may be distributed in different devices.

The present disclosure is not limited to the above embodiments, and can be appropriately modified without departing from the scope thereof. For example, in each of the above embodiments, the case where the trainee 900 is a hemiplegic patient suffering from paralysis in one leg has been described as an example, but the present disclosure is not limited to this. The trainee 900 may be, for example, a patient suffering from paralysis of both legs. In that case, the trainee 900 performs training while wearing the walking assist device 120 on both legs. Alternatively, the trainee 900 does not have to wear the walking assist device 120 on any of the legs.

Also in the above embodiment, the system control unit 200 measures the walking state of the trainee 900 based on the load distribution information output from the load distribution sensor 150 and the corrected image supplied from the information processing device 100. However, the system control unit 200 does not have to measure the walking state based on the load distribution information of the load distribution sensor 150.

What is claimed is:

1. A walking state measurement system comprising:
an endless belt that defines a walking surface on which a subject walks and that travels along a walking front-rear direction;
a marker provided on at least the walking surface of the belt along a circumferential direction of the belt;
a first camera for taking an image of the subject from front, the first camera taking an image of the marker on the walking surface from above to generate a first image;
a second camera for taking an image of the subject from front, the second camera taking an image of the marker on the walking surface from above to generate a second image; and
a correction processing unit that corrects an image generated by at least one of the first camera and the second camera based on a position of an image area of the marker included in each of the first image and the second image.

2. The walking state measurement system according to claim 1, wherein:
in the first image, pixel counts from both ends of the first image to both ends of the marker in a walking right-left direction are equal to or more than a first threshold value; and
in the second image, pixel counts from both ends of the second image to both ends of the marker in the walking right-left direction are equal to or more than a second threshold value.

3. The walking state measurement system according to claim 1, wherein:
in the first image, pixel counts from both ends of the first image to both ends of at least one marker in the walking front-rear direction are equal to or more than a third threshold value; and
in the second image, pixel counts from both ends of the second image to both ends of at least one marker in the walking front-rear direction are equal to or more than a fourth threshold value.

4. The walking state measurement system according to claim 1, wherein markers are arranged at an interval of a predetermined distance or more over an entire circumference of the belt.

5. The walking state measurement system according to claim 4, wherein the markers on the walking surface have different shapes or colors from each other.

6. The walking state measurement system according to claim 4, wherein the markers constitute a pattern with at least a half circumference of the belt being one cycle.

7. The walking state measurement system according to claim 1, wherein the marker is provided endlessly over an entire circumference of the belt.

8. A walking state measurement method comprising:
generating a first image by a first camera for taking an image of a subject from front, the first camera taking an image of a marker on a walking surface from above, the marker provided on at least the walking surface of a belt along a circumferential direction of the belt, the belt being an endless belt, the belt defining the walking surface on which the subject walks the belt traveling along a walking front-rear direction;
generating a second image by a second camera for taking an image of the subject from front, the second camera taking an image of the marker on the walking surface from above; and
correcting an image generated by at least one of the first camera and the second camera based on a position of an image area of the marker included in each of the first image and the second image.

9. A non-transitory computer readable medium storing a program that causes a computer to perform a walking state measurement method, wherein:
the method comprises
generating a first image by a first camera for taking an image of a subject from front, the first camera taking an image of a marker on a walking surface from above, the marker provided on at least the walking surface of a belt along a circumferential direction of the belt, the belt being an endless belt, the belt defining the walking surface on which the subject walks the belt traveling along a walking front-rear direction;
generating a second image by a second camera for taking an image of the subject from front, the second camera taking an image of the marker on the walking surface from above; and
correcting an image generated by at least one of the first camera and the second camera based on a position of an image area of the marker included in each of the first image and the second image.
